(19) United States
(12) Patent Application Publication
(10) Pub. No.: US 2003/0004141 A1
(43) Pub. Date: Jan. 2, 2003

(54) Medical devices, compositions and methods for treating vulnerable plaque

(76) Inventor: David L. Brown, Bronxville, NY (US)

Correspondence Address: OPPENHEIMER WOLFF & DONNELLY LLP, 840 NEWPORT CENTER DRIVE, SUITE 700, NEWPORT BEACH, CA, 92660, US

(21) Appl. No.: 10/096,131

(22) Filed: Mar. 8, 2002

Related U.S. Application Data
(60) Provisional application No. 60/274,331, filed on Mar. 8, 2001.

(51) Int. Cl. A61K 31/66; A61K 31/65; A61K 31/495; A61K 31/335; A61K 31/325; A61K 31/19; A61K 31/18

(52) U.S. Cl. 514/152; 514/252.12; 514/450; 514/478; 514/575; 514/588; 514/601; 514/137; 607/1

(57) ABSTRACT

Medical devices, compositions and methods for treating or preventing atherosclerotic plaque rupture are disclosed. Specifically, medical devices that deliver to a treatment site metalloproteinase inhibitors (MMPI) are disclosed. The medical devices include catheters, guide wires, vascular stents, micro-particles, electronic leads, probes, sensors, drug depots, transdermal patches, and vascular patches. Representative MMPIs included zinc chelators, urea derivatives, caprolactone-based inhibitors, phoshoinamides, piperazines, sulfonamides, tertiary amines, carbamate derivatives, mercaptoalcohols, mecaptoketones, antimicrobial tertracyclines, non-antimicrobial tetracyclines, and derivatives and combinations thereof. In one embodiment a self-expanding vascular stent is coated with at least one MMPI and deployed at a site within an artery where vulnerable plaque has been identified.

### RELATED APPLICATIONS

The present application claims priority to U.S. provisional patent application 60/274331 filed Mar. 8, 2001, now abandoned, the entire contents of which are herein by reference.

### FIELD OF THE INVENTION

The present invention relates to medical devices, compositions and methods for the prevention of acute coronary arty disease. Specifically, the present invention relates to methods and compositions used to stabilize vulnerable plaque thus preventing plaque rupture and the resulting microembolization. More specifically, the present invention relates to the use of metalloproteinase inhibitors to neutralize or suppress expression of methalloproteinases associated with vulnerable plaque rupture.

### BACKGROUND

Coronary Artery Disease (CAD) is a leading cause or death in nearly all developed countries. In the United States, the National Institutes for Health estimates that some form of CAD afflicts nearly 7 million Americans and that CAD is a primary cause of death in over 500,000 persons annually. Coronary artery disease is defined as a reduction of blood flow to the heart as a result of an occlusion in a coronary artery. Reduced blood flow to the heart, or ischemia, may be asymptomatic, chronic or acute. Over time, many asymptomatic persons develop chronic CAD beginning with mild chest pain (angina) while exerting and eventually leading to debilitating ischemia and persistent acute angina. However, in many cases, asymptomatic CAD can develop into acute coronary syndromes including unstable angina, myocardial infarction (MI) and even sudden death.

Both chronic and acute CAD result from atherosclerotic plaques formed on the artery's intimal layer (the innermost lining of the blood vessel composed of endothelial cells) in response to an injury. (P. K. Shah. 1997. Plaque Disruption and Coronary Thrombosis: New Insight into Pathogenesis and Prevention. Clin. Card. Vol. 20 (Suppl. II), II-38-II-44.) However, it is the type of atherosclerotic plaque formed that dictates whether the resulting CAD will be a stable chronic condition or acute CAD resulting in sudden death. (Id.) Atherosclerotic plaques are composed of a fibrous outer layer, or cap, and soft atheromatous core of fatty material referred to herein after as the atheromatous gruel. The exact composition of mature atherosclerotic plaques varies considerably and the factors that effect an atherosclerotic plaque's make-up are poorly understood. However, the fibrous cap associated with many atherosclerotic plaques is formed from a connective tissue matrix of smooth muscle cells, types I and III collagen and a single layer of endothelial cells. The atheromatous gruel is composed of blood-borne lipoproteins trapped in the sub-endothelial extracellular space and the breakdown of tissue macrophages filled with low density lipids (LDL) scavenged from the circulating blood. (G. Pasterkamp and E. Falk. 2000. Atherosclerotic Plaque Rupture: An Overview. J. Clin. Basic Cardiol. 3:81-86). The ratio of fibrous cap material to atheromatous gruel determines plaque stability and type.

There are two predominate populations of atherosclerotic plaques (Id). The plaque associated with stable chronic CAD is referred to as fibro-intimal lesions that are composed of fibrous tissue with minimal, if any atheromatous gruel. Unstable atherosclerotic plaque associated with acute CAD including unstable angina, myocardial infarction (MI) and even sudden death are lipid-laden lesions that have a soft central core and a thin fibrous cap (Id). Fibro-intimal plaques are generally quite stable and are associated with gradual luminal narrowing eventually leading to myocardial ischemia and anginal pain. These plagues are composed of 70% or more hard, collagen-rich sclerotic tissues are less likely to rupture. Consequently, survival rates associated with this type of plaque are generally good and the resulting ischemic heart disease is treated with vasodilators, angioplasty, and angioplasty with stenting or coronary bypass graft surgery. However, when a thick hard sclerotic cap does not support the atheromatous gruel rich core, the plague is subject to rupture. This type of plaque is referred to as vulnerable plaque and poses the greatest threat for acute CAD and sudden death (Id).

Atherosclerotic plaque forms in response to vascular endothelial cell injury associated with, among other causes, hyper-cholesterolemia, mechanical trauma, and autoimmune diseases. The injured endothelial cells secrete chemotactic and growth factors such as monocyte chemotactic protein 1 that cause circulating monocytes to converge on the injured site and attached to the endothelium. The monocytes then migrate into the sub-endothelium where they undergo a phenotypic transformation into tissue macrophages. The tissue macrophages begin scavenging LDL present in the blood ultimately forming foam cells and fatty streaks that eventually mature into atherosclerotic plaque (M. Navab, et al. 1991. Monocyte Transmission Induced by Modification of LDL in Co-culture of Human Aortic Wall Cells is Due to Induction of Monocyte Chemotactic Protein I Synthesis and Abolished by HDL. J. Clin. Invest. 88:2039-2040).

Plaque vulnerability is determined by a combination of intrinsic properties and extrinsic factors. The three most important intrinsic factors that predispose plaques to rupture include the size and consistence of the atheromatous core, thickness and collagen content of the fibrous cap, cap fatigue and inflammation. Atherosclerotic plaque begins to become increasing more unstable, and hence more vulnerable to rupture, when the lipid-laden core exceeds 40% of the total structure (B. Lundberg. 1985. Chemical Composition and Physical State of Lipid Deposits in Atherosclerosis. Atherosclerosis, 56:93-110). Furthermore, core composition is important in determining plaque vulnerability. Atherosclerotic gruel having increased amounts of extracellular lipids in the form of cholesterol esters (as opposed to cholesterol crystals) is particularly soft and increases plaque vulnerability. Moreover, inflammation and infection raise body temperature causing the plaque's cholesterol ester-rich gruel core temperature to increase. As the core warms it becomes increasingly unstable and susceptible to rupture.

The second intrinsic factor affecting plaque vulnerability is cap thickness and content. Cap cellularity, matrix composition and collagen content varies considerably (M. J. Davis, et al. 1993. Risk of Thrombosis in Human Atherosclerotic Plaques: Role of Extracellular Lipid, Macrophages and Smooth Muscle Cell Content. Br. Heart J. 69:377-381). Generally, caps having fewer collagen synthesizing cells are inherently weaker than caps with higher collagen content. Collagen content determines a cap's tensile strength, especially at the junction between the plaque and adjacent vessel wall. The region, referred to as the plaque shoulder, is often the thinnest and most heavily infiltrated with macrophages and foam cells. Consequently, the plaque shoulder region is inherently unstable the site were rupture usually occurs.

Recently, inflammation has been identified as a potential factor in plaque rupture leading to acute coronary syndromes (E. Falk, et al. 1995. Coronary Plaque Disruption. Circulation, 92:657-671). Disrupted fibrous caps taken post mortum from patients with unstable angina are often more heavily infiltrated with macrophages at the plaque rupture site than plaque from cases of stable angina. In addition to macrophages, other cells involved in the inflammatory response are also found in atherosclerotic plaque. T lymphocytes, mast cells and neutrophils secrete cytokine and protolytic enzymes that contribute to plaque instability. Activated T-cells infiltrate the plaque and compromise plaque structural integrity by secreting interferon-γ (INF-γ) which in turn down regulates collagen synthesis within the fibrous cap, inhibits vascular smooth muscle cell (VSMC) proliferation and induces VSMC apoptosis. Furthermore, INF-γ also activates tissue macrophages present in the lesion as well as circulating macrophages (P. R. Moreno, et al. 1996. Macrophages, Smooth Muscle Cells, and Tissue Factor in Unstable Angina. Implications for Cell-Mediated Thrombogenicity in Acute Coronary Syndromes. Circulation. 94: 3090-3097). Activated macrophages secrete protolytic proteins that degrade the caps extracellular matrix decreasing cap thickness as well as increasing macrophage infiltration which contributes to gruel mass and shoulder instability. Recently, a group of proteolytic enzymes known as matrix metalloproteinases have been shown to attack and degrade the fibrillar interstitial collagen characteristic of plaque caps. (G. K. Sukhova, et al. 1999. Evidence for Increased Collagenolysis by Interstitial Collagenases-1 and -3 in Vulnerable Human Atheromatous Plaques. Circulation; 99:2503-2509; see also Z. Galis, et al. 1994. Increased Expression of Matrix Metalloproteinases and Matrix Degrading Activity in Vulnerable Regions of Human Atherosclerotic Plaques. J. Clin. Invest.; 94: 2493-2503; see also C. M. Dollery, et al. 1995. Matrix Metalloproteinases and Cardiovascular Diseases. Circ. Res.; 77:863-868).

Atherosclerotic plaques are structures within or adjacent to the arterial wall that are subjected to a number of extrinsic factors that trigger plaque rupture. These extrinsic factors are same physical stresses endured by the arterial wall itself including circumferential force, compressive forces, circumferential bending, longitudinal flexion and hemodynamic forces. Circumferential forces within a vessel lumen are determined by blood volume, blood pressure and lumen diameter. Circumferential pressure increases as blood volume and pressure increase. The narrower the vessel lumen, the greater the circumferential pressure will be for any given blood volume or pressure. Circumferential forces exert pressure against the vessel wall which is resisted by the circumferential tension. Without circumferential tension, the vessel wall would continue to expand until aneurysm results. However, the circumferential tension is not exerted by the vessel wall exclusively, vessel wall structures such as plague also exert tension in response to the circumferential forces (A. Maclssac, et al. 1993. Toward the Quiesent Coronary Plaque. J. Am. Coll. Cardiolo., 22:1228-1241).

Plaques associated with stable CAD have thick fibrous caps and minimal soft atheromatous core. Consequently, as circumferential force increases within the vessel the resulting circumferential tension is distributed throughout the thick fibrous cap with minimal load bearing being done by the soft gruel. As a result the lesion remains stable and resists rupture. However, as the gruel content increases and cap thickness decreases, circumferential tension cannot be adequately dissipated the fibrous cap and increased pressure from the lumen is exerted on the soft atheromatous core. Once this pressure reaches a critical point the cap ruptures, usually at the shoulder region.

Fibrous cap compression is essentially the opposite of circumferential force. Circumferential force results from tension created as the vessel lumen resists expansion. The greater the pressure within the lumen, the greater the circumferential tension that must be applied to resist aneurysm. As the tension mounts within the lumen wall, it is communicated directly to the interior of attached structures such as plaque. Consequently, the greater the circumferential force, the greater the pressures become against the plaque core. As previously explained, plaques having a higher fibrous cap to soft atheromatous core ratio are better able to distribute the luminal pressure and resist rupturing. Plaque compression is often results from vasospasm where the lumen wall presses against attached these structures compressing the plaque core. Plaques having a greater volume of soft atheromatous core and a thin fibrous cap are most prone to compression rupture (R. T. Lee and R. D. Kamm. 1994. Vascular Mechanics for the Cardiologist. J. Am. Coll. Cardiol. 23; 1289-1295).

Other extrinsic mechanical factors such as circumferential bending and longitudinal flexion are less important than cap tension and compression in plaque rupture. Circumferential bending is caused by the normal pulse wave generated within the vessel lumen associated with changes in luminal blood pressure. During the diastolic-systolic cycle the lumen diameter will change approximately 10 percent (Id). This constant fluctuation in lumen diameter results in circumferential bending of the atherosclerotic plaque. Longitudinal flexion results form the normal beating of the heart. Coronary arteries anchored to the myocardium are constantly stretched and relaxed as the heartbeats. This exerts a longitudinal stress on the vessel lumen which is directly communicated to attached structures such as atherosclerotic plaque. The combined actions of circumferential bending and longitudinal flexing exert forces on the plaque fibrous cap as described above. Thus, the thicker the cap, the more resistant to rupture the plaque becomes (Id).

The last extrinsic force, hemodynamic factors are non-mechanical in nature and probably contribute the least to plaque rupture. Hemodynamic forces are generally associated with shear stress. Shear force result from turbulence created as a fluid change velocity in response to topological changes in the arterial wall (M. L. Armstrong, at al. 1985. Structural and Hemodynamic Responses to Peripheral Arteries of Macaque Monkeys to Atherosclerotic Diet. Arteriosclerosis. 5:336-346). For example, blood flowing through an artery having a fixed diameter moves at a constant speed. However, when the blood flow reaches a stricture in the vessel caused by plaque, it accelerates through the narrowing consistent with Bernoulli's principle. As the blood flow passes the narrowed lumen region it slows creating vortices in the blood flow that can theoretically disrupt the plaque. Obviously, stable plaques having thick caps will be less affected than plaques with thin caps and large volumes of atheromatous gruel.

Regardless of the cause, once plaque rupture occurs, thrombus formation is initiated. Rupture of the lipid-laden plaque exposes the highly thrombogenic atheromatous core and the sub-endothelium VSMC component of the arterial wall to the circulation. Platelet aggregation and adherence to the sub-endothelium follow this almost immediately. Platelet adhesion results in their activation and release of growth factors into the circulating blood and the initiation of the coagulation cascade. The released growth facts, specifically platelet-derived growth factor (PDGF) stimulates the proliferation and migration of VSMC. Proliferation and migration of VSMC can lead to plaque remodeling and increased vascular stenosis, or interact with the platelets leading to enhanced thrombogenesis (G. Pasterkamp and E. Falk. 2000. Atherosclerotic Plaque Rupture: An Overview. J Clin. Basic Cardiol. 3:81-86).

The extent of vascular injury following plaque rupture determines the platelet adherence rates and thrombus formation. Platelet adherence and thrombus formation is complete within five to ten minutes when the injury to the vessel intima is superficial. The resulting thrombus is relatively unstable and is easily dislodged by blood flow shear forces. Once dislodged, the thrombus can be carried down stream causing unstable angina, MI or strokes (L. Badimon, et al. 1986. Influence of Arterial Wall Damage and Wall Sheer Rate on Platellet Deposition: Ex vivo Study in Swine Model. Arteriosclerosis. 6:312). Deep vessel injury results in enhanced platelet deposition and thrombus formation that is located deeper within the intimal or medial layers. These thrombi are less easily dislodged but can contribute to abrupt arterial occlusion and sudden death. However, regardless of the magnitude of vessel injury, once the coagulation cascade has been initiated, thrombi formed in the heart's vasculature present significant short and long term health risks (V. Fuster, et al. 1988. Insights into the Pathogenesis of Acute Ischemic Syndromes. Circulation. 77:1213-1220).

Stable plaques have minimal atheromatous gruel, thick caps, are relatively stable and generally do not present a risk of MI or sudden death. Stable plaques will most probably either result in progressive ischemic CAD or remain asymptomatic for life. However, as discussed above, vulnerable plaque can result in life threatening CAD including sudden death. Coronary artery disease associated with stable plaque can be effectively treated using minimally invasive procedures including angioplasty, stenting or medications. However, satisfactory acute therapies for treating vulnerable plaque are extremely limited.

Recently, new techniques have been developed that permit vulnerable plaque detection and risk assessment using a percutaneous procedure. Therefore, it would be a significant advance to the treatment of CAD if methods were developed for treating vulnerable plaque coincident with detection. One method for treating vulnerable plaque would be to stabilize the lesion through cap reinforcement, atheromatous gruel volume reduction or combinations thereof. Lipid lowering therapy may reduce the risks associated with vulnerable plaque by reducing its lipid content. (D. M. Small. 1988. Progression and Regression of Atherosclerotic Lesions. Insights from Lipid Physical Biochemistry. Atheroscl. 8;103-1029.) However, most lipid lowering regimens may require many months or years of use to significantly reduce the risk of MI or sudden death. (H. M. Loree et al. 1994. Mechanical Properties of Model Atherosclerotic Lesion Lipid Pools. Arterioscl. Thromb. 14:230-234.) Therefore, immediate plaque stabilization therapies will focus on cap reinforcement techniques. One particular attractive therapeutic target conducive to immediate intervention therapy is neutralizing cap disintegrating proteinases secreted by activated macrophages. These proteinases undermine the cap's structural integrity by digesting the fibrous networks associated with stable plaque. Therefore, methods and technologies designed to inhibit proteinase expression or neutralize expressed enzymes may play a major role in preventing the most serious forms of CAD associated with vulnerable plaque.

### SUMMARY OF THE INVENTION

The present invention relates to methods and compositions used to inhibit vulnerable plaque rupture by neutralizing or inhibiting plaque cap-weakening protolytic enzymes. Specifically, the present invention relates to neutralizing or inhibiting metalloproteinases secreted by vulnerable plaque associated activated macrophages. More specifically, the present invention is directed at inhibiting matrix metalloproteinases (MMP) responsible for the break-down of fibrillar interstitial collagen characteristic of the vulnerable plaque's fibrous caps.

In one embodiment of the present invention MMP inhibitors (MMPI), or combinations thereof are delivered in situ to an area in need of treatment using a medical device selected from the group consisting of catheters, guide wires, vascular stents, micro-particles, electronic leads, probes, sensors, drug depots, transdermal patches, vascular patches and other implantable medical devices.

In one embodiment of the present invention the treatment area in need of treatment comprises a blood vessel lumen, specifically an arterial lumen.

In another embodiment of the present invention the MMPI composition is dispersed in a biocompatible polymer that is used to form or coat an implantable medical device. In one embodiment of the present invention the medical device is a woven vascular stent. In one embodiment the monofilaments used to form the woven vascular stent comprise poly-L-lactide, in another embodiment, polycaprolactam, in yet another embodiment the monofilaments are a mixture of poly-L-lactide and caprolactam.

Other embodiments may include polymeric MMPI releasing depots that are responsive to thermal energy or electrical current. In either case the polymeric matrix is effected resulting in an increased rate of delivery of the MMPI composition sequestered within the polymeric depot. The polymeric depots of the present invention include, but are not limited to, polymeric stents, stent coatings, coated probes, catheters, and microparticles.

In yet another embodiment of the present invention the MMPI impregnated biocompatible polymer is used to form microparticles that are injected to the treatment area by catheter, or delivered trans-myocadially into the pericardial space. In another embodiment a transdermally implanted drug delivery depot is made from the MMPI impregnated biocompatible polymer.

Other embodiments of the present invention include surgical patches and transdermal patches impregnated with biocompatible hydrogels having the MMPI composition dispersed therein. The patch is placed on or near the treatment area and the MMPI composition passively diffuses into the treatment site.

The MMPI compositions of the present invention include, but are not limited to, zinc chelators, urea derivatives, caprolactone-based inhibitors, phoshoinamides, piperazines, sulfonamides, tertiary amines, carbamate derivatives, mercaptoalcohols, mecaptoketones, antimicrobial tertracyclines, non-antimicrobial tetracyclines, and derivatives and combinations thereof.

Additional embodiments of the present invention will be apparent to those skilled in the art from the detailed disclosure that follows.

### DETAILED DESCRIPTION

Prior to setting forth the invention, it may be helpful to an understanding thereof to set forth definitions of certain terms that will be used hereinafter.

Atherosclerosis: The insudation of fatty substances and fibrous proteins that make up the atherosclerotic plaque of arteries.

Atheromatous: Fatty degeneration of an artery's initimal lining.

Atheromatous gruel: A mixture of lysed vascular smooth muscle cells, endothelial cells, blood cells, intact macrophages, cholesterol and low density lipids found in the core of atherosclerotic plaque.

Atheromatous core: See atheromatous gruel above.

MMPI composition: One or more Metalloproteinase inhibitors demonstrated to be efficacious in treating vulnerable plaque and dispersed or dissolved in a suitable carrier or solvent.

Vulnerable Plaque: Includes atherosclerotic plaque that is at risk for rupture. Although vulnerable plaque as used herein generally refers to plaque having a significant lipid pool, pre-plaque can also be at risk for rupture and will be included in the definition of “vulnerable plaque” throughout the specification and claims. Moreover, the methods and compositions of the present invention may also be used to for inhibiting rupture of plaque normally considered to be “stable.”

Plaque rupture and the resulting throbogenesis is a leading cause of sudden cardiovascular arterial disease (CAD) associated death in developed countries. There are essentially two categories of plaques found in the human vasculature: fibro-intimal plaques associated with stable, slowly progressive or benign CAD, and vulnerable plaque which is associated with unstable angina, myocardial infarction (MI) and sudden death. Fibro-intimal plaque has a well developed, thick fibrous cap and small soft atheromatous core. Vulnerable plaque has a thinner, less well developed fibrous cap and a larger soft core composed of an atheromatous gruel.

Vulnerable plaque is physically unstable and prone to spontaneous rupture when exposed to normal physiological factors such as changes in arterial blood pressure (circumferential forces), the normal rhythmic beating of the heart (longitudinal flexion) and blood flow related hemodynamic forces among others. The most important factors that predispose plaque to spontaneous rupture include the thickness of the fibrous cap, the relative ratio of the cap to the soft atheromatous gruel-core and the physical integrity of the cap itself.

As previous discussed, vulnerable plaque has a thinner cap and higher atheromatous gruel content than fibro-inimal, or stable, plaque. However, it has been shown that the cap's fibrillar interstitial collagen content and integrity is a primary factor that predisposes vulnerable plaque to rupture. Collagen is a complex group of fibrous structural proteins that form the main component of animal-derived connective tissues. Collagen is the most abundant protein in animals and is the primary protein component of skin, bones, tendons, cartilage, blood vessels and teeth. Virtually every cell of the body including vascular smooth muscle cells (VSMC) secretes collagen in one or more of its various forms. Collagen synthesis begins intracellularly with the production of a triple helix composed of three polypeptide strands. The pro-collagen is then secreted into the extracellular spaces of the connective tissue where post secretion modification, molecular aggregation and cross-linking forms mature collagen. Mature collagen is a triple helix composed of three polypeptide chains. In mammals, over 30 discrete polypeptide chains have been identified comprising the 16 collagen variants distributed through an animals' tissues. The most abundant types of collagen include Types I, II and III. Type I collagen is found in skin, bone, tendon, cornea, and blood vessels; Type II is distributed in the cartilage and intervertebral disks; Type III is a competent of fetal skin and blood vessels. The three polypeptide chains that comprise a collagen molecule may be the same, as in the case of type II and III collagen, or two different polypeptide chains may be used to form the mature triple helix. In the case of atherosclerotic plaque development, types I and III collagen are secreted by VSMC in response to growth factors secreted by platelets, macrophages, and damaged endothelial cells at the site of the initial vascular injury. It is this VSMC secreted collagen that makes up the fibrous cap's primary structural components.

Collagens are highly stable proteins whose metabolic break down and readsorption (catabolism) is mediated by a variety of proteolytic enzymes. One of the most important group of proteolytic enzymes responsible for collagen catabolism is matrix metalloproteinases (MMP). The MMPs are a diverse family of zinc- and calcium-dependent enzymes that include collagenases, gelatinases, stromelysin, and membrane-type MMPs. These enzymes are secreted by macrophages, lymphocytes and smooth muscle cells sequestered within the atherosclerotic plaque and collectively catabolize substrates responsible for maintaining the structural integrity of the fibrous cap. These fibrous cap structural substrates include collagen, elastin, proteoglycan, laminin, fibronectin and cell basement membrane components (gelatin). Table 1 below lists the most common MMPs and their respective substrates. Traditionally, enzymes have been named for the dominant substrate involved in biological reactions they catalyze. However, many enzymes, including MMPs have affinity for a number of different substrates. Therefore, naming enzymes after their respective substrates often leads to confusion. To overcome this problem biological chemists have given each enzyme a unique identification number. In the case of the MMPs each enzyme has been assigned a unique “MMP” number. In addition to “MMP” numbers, the Enzyme Commission, or EC, has also assigned each MMP a unique number. For example, Collagenase I has been designated EC3.4.24.7. Unfortunately, the Enzyme Commission has lagged behind in designating each MMP an identifier. Consequently, there remains confusion in the literature.

It is important to note that the MMP numbers are not necessarily sequential. For example, here is no MMP-4, -5 or -6. Moreover, more recently identified MMPs have not been given common names. It should also be understood that the list of enzymes and their substrates given in Table I is not inclusive. Only the MMPs and their substrates most relevant to vulnerable plaque weakening have been listed. For additional information on MMPs, see: J. Frederick Woessner and Hideaki Nagase. Matix Metalloproteinases and TIMPs, Oxford University Press; 2000.

TABLE 1
Representative Matrix Metalloproteinases Involved in Vulnerable Plaque
Weakening

| MMP No | | |
|---|---|---|
| EC No | Common Name | Enzyme Substrate |
| 1 | Collagenase 1 | Collagens I, II, III, VII, VIII and IX, |
| EC3.4.24.7 | Fibroblast | proteoglycan |
| | collagenase | |
| | Interstitial | |
| | collagenase | |
| 2 | Gelatinase A | Collagens I, IV, VII, X, XI and XIV, |
| EC3.4.24.24 | 72-kDa Gelatinase | gelatin, elastin, fibronectin, |
| | | proteoglycan |
| 3 | Stromelysin 1 | Collagens III, IV, V, IX, gelatin, |
| EC3.4.24.17 | | elastin, fibrinogen |
| 8 | Collagenase 2 | Collagens I, II, III, V, VII, VIII, X, |
| EC3.4.24.34 | Neutrophil | gelatin, fibronectin. |
| | collagenase | |
| 9 | Gelatinase B | Collagens IV, V, VII, X, XIV, |
| EC3.4.24.35 | 92-kDa Gelatinase | gelatin, elastin, fibronectin. |
| 10 | Stromelysin 2 | Collagens III, IV, V, gelatin, elastin, |
| EC3.4.24.22 | | proteoglycan |
| 11 | Stromelysin 3 | Laminin, fibronectin, gelatin, |
| EC # not | | Collagen IV |
| assigned | | |
| 12 | Macrophage elastase | Collagen IV, gelatin, elastin, laminin, |
| EC # not | | proteoglycan. |
| assigned | | |
| 13 | Collagenase 3 Rat | Collagens I, II, III, IV, IX, X, XIV, |
| EC # not | osteoblast | gelatin, fibronectin |
| assigned | collagenase | |
| 14 | MT1-MMP | Collagen I, II, III, gelatin, |
| EC # not | | fibronectin, laminin, proteoglycan |
| assigned | | |
| 15 | MT2-MMP | Fibronectin, laminin |
| EC # not | | |
| assigned | | |
| 16 | MT3-MMP | Collagen III, gelatin, fibronectin |
| EC # not | | |
| assigned | | |
| 17 | MT4-MMP | Gelatin |
| EC # not | | |
| assigned | | |
| 18 | Collagenase 4 | Collagen IV |
| EC # not | | |
| assigned | | |

When cells sequestered within the atherosclerotic plaque secrete MMPs into the extracellular milieu these proteolytic enzymes begin breaking down the fibrous collagen cap thereby increasing the risk of plaque rupture. Therefore, the present inventor has recognized the need for methods and compositions that neutralize MMP activity or inhibit their secretion (collectively referred to herein after as MMP inhibitors, or MMP). Specifically, the present inventor has recognized the need for methods and devices for the site specific delivery of MMPIs.

Matrix metalloproteinases are recognized has having an important role in tumor growth and progression. Human clinical trials designed to establish the efficacy of MMPls such as marimastat are presently ongoing. Marimastat is a hydroxamic acid derivative related to the broad-spectrum MMPI batimastat. Batimastat and its analogue marimastat are zinc chelators that react with the MMP's zinc center and complex with the enzyme (A. H. Drummond, et al. 1999. Pre-clinical and Clinical Studies of MMP Inhibitors in Cancer. Annals of the New York Academy of the Sciences, Jun 30;878:228-235). The complexed MMP can no longer bind to its intended substrate thus effectively inhibiting the MMP's proteolytic activity (Id). Furthermore, MMPIs such as batimastat, marimastat and others exhibit minimal in vivo toxicity (Id). Consequently, MMPIs represent a particularly promising group of therapeutic compounds.

More recently, a significant effort has been directed at designing new MMPIs having efficacy and toxicological profiles similar to marimastat (D. E. Biswanath, et al. 1999. The next Generation of MMP Inhibitors, Design and Synthesis. Annals of the New York Academy of the Sciences, Jun 30;878:40-60). The most promising candidates have been urea derivatives, caprolactone-based inhibitors, phoshoinamides. piperazines, sulfonamides, tertiary amines, carbamate derivatives, mercaptoalcohols, mecaptoketones and derivatives thereof (Id). Another group of promising MMPI include antimicrobial and non-antimicrobial tetracyclines including, CMT-8, tetracycline, doxycycline and minocycline. (L. M. Golub, et al. 1999. A Chemically Modified Non-antimicrobial Tetracycline (CMT-8) Inhibits Gingival Matrix Metalloproteinases, Periodontal Breakdown, and Extraoral Bone Loss in Ovariectomized Rats. Annals of the New York Academy of the Sciences, Jun 30;878:290-310).

Matrix metalloproteinases are involved in numerous essential metabolic processes including, tissue remolding, normal cell migration, and protein processing including enzyme activation, post transcriptional protein modifications, protein turn-over and fragment generation. In vivo, MMPI activity is closely regulated. Matrix metalloproteinases contain a highly conserved proteinase domain having three histidine residues that form a complex with catalytic zinc ion. Furthermore, MMPs have a conserved regulatory domain that bind cysteine residues to the zinc active site thus keeping the MMP in an inactive form until needed. Once activated, MMPs demonstrate substrate specificity primarily through the varying topologies of the active site clefts within their catalytic domains. Differences in other molecular domains further contribute substrate specificity and determine interactions with the body's natural MMP inhibitors, Tissue Inhibitors of Metalloproteinases (TIMPs).

The present invention is directed at the site specific delivery of synthetic MMPIs and TIMPs to suppresses MMP activity in vulnerable plaque. Using the teachings herein, and combined with teaching known to those in the art, the skilled practitioner will be able to ascertain the MMP inhibition spectrum for a given compound and will select the MMPIs necessary to inhibit the MMPs associated with vulnerable plaque rupture. (See, for example: J. S. Skotnicki, et al. 1999. Design and Synthetic Considerations of Matrix Metalloproteinase Inhibitors. Annals of the New York Academy of the Sciences, Jun 30;878:62-72 and D. E. Biswanath, et al. 1999, The next Generation of MMP Inhibitors, Design and Synthesis. Annals of the New York Academy of the Sciences, Jun 30;878:40-60.) Finally, the MMP distribution within vulnerable plaque has been discussed in detail herein and the skilled artisan will also consult references such as, but not limited to G. K. Sukhova, et al. 1999. Evidence for Increased Collagenolysis by Interstitial Collagenases-1 and -3 in Vulnerable Human Atheromatous Plaques. Circulation; 99:2503-2509; see also Z. Galis, et al. 1994. Increased Expression of Matrix Metalloproteinases and Matrix Degrading Activity in Vulnerable Regions of Human Atherosclerotic Plaques. J. Clin. Invest.; 94:2493-2503; see also C. M. Dollery, et al. 1995. Matrix Metalloproteinases and Cardiovascular Diseases. Circ. Res.; 77:863-868.

In addition to MMPI selection, treatment efficacy may also be effected by factors including dosage, route of delivery and the extent of the disease process (treatment area). An effective amount of a MMPI composition can be ascertained using methods known to those having ordinary skill in the art of medicinal chemistry and pharmacology. First the toxicological profile for a given MMPI composition is established using standard laboratory methods. For example, the candidate MMPI composition is tested at various concentration in vitro using cell culture systems in order to determine cytotoxicity. Once a non-toxic, or minimally toxic, concentration range is established, the MMPI composition is tested throughout that range in vivo using a suitable animal model. After establishing the in vitro and in vivo toxicological profile for the MMPI composition, it is tested in vitro to ascertain the compound retains potentially efficacious MMP inhibition at the non-toxic, or minimally toxic ranges established.

Finally, the candidate MMPI composition is administered to treatment areas in humans in accordance with either approved Food and Drug Administration (FDA) clinical trial protocols, or protocol approved by Institutional Review Boards (IRB) having authority to recommend and approve human clinical trials for minimally invasive procedures. Treatment areas are selected using vulnerable plaque detection methods and compositions such as those disclosed in U.S. Pat. No. 5,871,449 issued to the present inventor on Feb. 16, 1999. The candidate MMPI composition is then applied to the selected treatment areas using a range of doses. Preferably, the optimum dosages will be the highest non-toxic, or minimally toxic concentration established for the MMPI composition being tested. Clinical follow-up will be conducted as required to monitor treatment efficacy and in vivo toxicity. Such intervals will be determined based on the clinical experience of the skilled practitioner and/or those established in the clinical trial protocols in collaboration with the investigator and the FDA or IRB supervising the study.

The MMPI therapy for vulnerable plaque of the present invention can be administered directly to the treatment area using any number of techniques and/or medical devices. In one embodiment of the present invention the MMPI composition is applied to a vascular stent. The vascular stent can be of any composition or design. For example, the sent may be self-expanding or mechanically expanded using a balloon catheter. The stent may be made from stainless steel, titanium alloys, nickel alloys or biocompatible polymers. Furthermore, the stent may be polymeric or a metallic stent coated with at least one polymer. In another embodiments the delivery device is an aneurysm shield, a vascular graft or surgical patch. In yet other embodiments the MMPI therapy of the present invention is delivered using a porous or “weeping” catheter to deliver an MMPI containing hydrogel composition to the treatment area. Still other embodiments include microparticles delivered using a catheter or other intravascular or transmyocardial device.

The medical device can be made of virtually any biocompatible material having physical properties suitable for the design. For example, tantalum, stainless steel and nitinol have been proven suitable for many medical devices and could be used in the present invention. Also, medical devices made with biostable or bioabsorbable polymers such as poly(ethylene terephthalate), polyacetal, poly(lactic acid), poly(ethylene oxide)/poly(butylene terephthalate) copolymer could be used in the present invention. Although the medical device surface should be clean and free from contaminants that may be introduced during manufacturing, the medical device surface requires no particular surface treatment in order to retain the coating applied in the present invention. Both surfaces (inner and outer, or top and bottom depending on the medical devices' configuration) of the medical device may be provided with the coating according to the present invention.

In order to provide the coated medical device according to the present invention, a solution which includes a solvent, a polymer dissolved in the solvent and a MMPI composition dispersed in the solvent is first prepared. It is important to choose a solvent, a polymer and a therapeutic substance that are mutually compatible. It is essential that the solvent is capable of placing the polymer into solution at the concentration desired in the solution. It is also essential that the solvent and polymer chosen do not chemically alter the MMPI's therapeutic character. However, the MMPI composition only needs to be dispersed throughout the solvent so that it may be either in a true solution with the solvent or dispersed in fine particles in the solvent. The solution is applied to the medical device and the solvent is allowed to evaporate leaving a coating on the medical device comprising the polymer(s) and the MMPI composition.

Typically, the solution can be applied to the medical device by either spraying the solution onto the medical device or immersing the medical device in the solution. Whether one chooses application by immersion or application by spraying depends principally on the viscosity and surface tension of the solution, however, it has been found that spraying in a fine spray such as that available from an airbrush will provide a coating with the greatest uniformity and will provide the greatest control over the amount of coating material to be applied to the medical device. In either a coating applied by spraying or by immersion, multiple application steps are generally desirable to provide improved coating uniformity and improved control over the amount of MMPI composition to be applied to the medical device. The total thickness of the polymeric coating will range from approximately 1 micron to about 20 microns or greater. In one embodiment of the present invention the MMPI composition is contained within a base coat, and a top coat is applied over the MMPI containing base coat to control release of the MMPI into the tissue.

The polymer chosen must be a polymer that is biocompatible and minimizes irritation to the vessel wall when the medical device is implanted. The polymer may be either a biostable or a bioabsorbable polymer depending on the desired rate of release or the desired degree of polymer stability. Bioabsorbable polymers that could be used include poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid.

Also, biostable polymers with a relatively low chronic tissue response such as polyurethanes, silicones, and polyesters could be used and other polymers could also be used if they can be dissolved and cured or polymerized on the medical device such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The polymer to MMPI composition ratio will depend on the efficacy of the polymer in securing the MMPI composition onto the medical device and the rate at which the coating is to release the MMPI composition to the tissue of the blood vessel. More polymer may be needed if it has relatively poor efficacy in retaining the MMPI composition on the medical device and more polymer may be needed in order to provide an elution matrix that limits the elution of a very soluble MMPI composition. A wide ratio of therapeutic substance to polymer could therefore be appropriate and could range from about 10:1 to about 1:100.

In one embodiment of the present invention a self-expanding nitinol stent is coated with MMPIs using a two-layer polymeric matrix. The base layer comprises a solution of ethylene-co-vinylacetate and polybutylmethacrylate. The MMPI or mixture thereof is incorporated into the base layer. The outer layer comprises only polybutylmethacrylate and controls that rate at which the MMPIs elute from the medical device. Briefly, the thickness of the polybutylmethacrylate outer layer determines the rate at which the MMPIs elute from the base coat by acting as a diffusion barrier. The ethylene-co-vinylacetate, polybutylmethacrylate and MMPI solution may be incorporated into or onto the medical device in a number of ways. In one embodiment of the present invention the MMPI/polymer solution is sprayed onto the medical device and then allowed to dry. In another embodiment, the solution may be electrically charged to one polarity and the medical device electrically changed to the opposite polarity. In this manner, the MMPI/polymer solution and medical device will be attracted to one another thus reducing waste and proving more control over the coating thickness.

In another embodiment of the present invention the medical device is coated with a polymeric composition (or composed entirely of a polymeric composition) that can be stimulated, either directly or remotely, to release a predetermined amount of MMPI composition. For example, it has recently been reported that ultrasound energy increases the permeability of skin to proteins by disorganizing the highly organized, impermeable structure of the lipid by-layers of the stratum coreum (S. Mitragotri, et al, 1995. Ultrasound-mediated Transdermal Protein Delivery. Science; 269:850-853). Based on this finding C. S. Kwok et al. proposed the use of ultrasound energy to distort tightly packed surface structures immobilized on polymer surfaces (C. S. Kwok, et al. 2001. Self-assembled Molecular Structures as Ultrasonically-responsive Barrier Membranes for Pulsatile Drug Delivery. J. Bio. Mat. Res.; 57:2:151-164).

The present inventors have applied this principle to the site specific controlled delivery of MMPI compositions. In one embodiment of the present invention a MMPI composition delivery polymer depot is prepared by polymerizing 2-hydroxyethyl methacrylate (HEMA) monomer and crosslinking the resulting polymer with triethyleneglycol dimethacrylate (TEGDM). In one embodiment of the present invention the pHEMA film is formed on the surfaces of a nitinol self-expanding vascular stent.

The pHEMA polymer composition made in accordance with the teachings of the present invention comprises of approximately 55 to 60 percent HEMA, 2 to 3 percent TEGDM, 15 to 20 percent ethylene glycol and the remain percentage being made up of water. The MMPI composition is added to the resulting polymer in a concentration of between approximately 3 to 5 percent depending on the MMPI employed. Other variation in the polymer matrix are also possible to optimize the controlled release of the MMPI composition. Finally, ordered methylene chains are prepared on the surface of the MMPI containing pHEMA substrate. The procedures used to form the ordered methylene chains are identical as those decried by C.S. Kwok, et al. 2000. Surface Modification of Polymeric Slabs with Self-Assembled Monolayer and its Characterization with multi-surface-analytical Techniques. Biomacromolecules; 1:139-148.

The resulting self-expanding vascular stent having the MMPI pHEMA coating described above is implanted into an patient at a treatment site where vulnerable plaque has been previously identified. Next an ultrasound frequency between is applied to the pHEMA/MMPI coated stent to regulate the MMPI release at the treatment site using techniques known to those skilled in the art of intravascular ultrasound techniques. The amount of MMPI release is directly proportional to the amount of ultrasound energy applied and the duration of exposure.

Other embodiments may include polymeric MMPI releasing depots that are responsive to thermal energy or electrical current. In either case the polymeric matrix is effected resulting in an increased rate of delivery of the MMPI composition sequestered within the polymeric depot. The polymeric depots of the present invention include, but are not limited to, polymeric stents, stent coatings, coated probes, catheters, and microparticles

In another embodiment of the present invention the MMPI composition is dispersed in a biocompatible polymer that is used to form an implantable medical device. In one embodiment of the present invention the medical device is a woven vascular stent. In one embodiment the monofilaments used to form the woven vascular stent comprise poly-L-lactide, in another embodiment, polycaprolactam, in yet another embodiment the monofilaments are a mixture of poly-L-lactide and caprolactam.

In yet another embodiment of the present invention the MMPI impregnated biocompatible polymer is used to form microparticles that are injected to the treatment area by catheter, or delivered trans-myocadially into the pericardial space. In another embodiment a transdermally implanted drug delivery depot is made from the MMPI impregnated biocompatible polymer.

Other embodiments of the present invention include surgical patches and transdermal patches impregnated with biocompatible hydrogels having the MMPI composition dispersed therein. The patch is placed on or near the treatment site and the MMPI compositions are delivered to the vulnerable plaque by diffusion.

As is evident form the foregoing detailed description, there are many methods and compositions that can be used to treat vulnerable plaque. Depending on the extent of the treatment area and the severity of the disease process, one, or more methods or compositions can be used. Therefore, it will be appreciated by those skilled in the art that while the invention has been described above in connection with particular embodiments, the invention is not necessarily limited to and that numerous modifications and departures from the embodiments, examples and uses may be made without departing from the inventive concepts.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Of course, variations on those preferred embodiments will become apparent to those of ordinary skill in the art upon reading the foregoing description. The inventor expects skilled artisans to employ such variations as appropriate, and the inventor intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Furthermore, numerous references have been made to patents and printed publications throughout this specification. Each of the above cited references and printed publications are herein specifically and individually incorporated by reference.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method of treating vulnerable plaque within a patent comprising:
a) detecting a site of vulnerable plaque in the patient; and
b) delivering at least one metalloproteinase inhibitor (MMPI) composition to said site.

2. The method according to claim 1 wherein said MMPI is selected from the group consisting of zinc chelators, urea derivatives, caprolactone-based inhibitors, phoshoinamides, piperazines, sulfonamides, tertiary amines, carbamate derivatives, mercaptoalcohols, mecaptoketones, antimicrobial tertracyclines, non-antimicrobial tetracyclines, and derivatives and combinations thereof.

3. The method according to claim 2 wherein said antimicrobial tetracycline is selected form the group consisting of tetracycline, doxycycline and minocycline.

4. The method according to claim 2 wherein said non-antimicrobial tetracycline CMT-8.

5. The method according to claim 2 wherein said zinc chelator is a hydroxamic acid derivative.

6. The method according to claim 5 wherein said hydroxamic acid derivative is marimastat or batimastat.

7. The method according to claim 1 wherein said MMPI is a naturally occurring tissue inhibitor of metalloproteinases (TIMP) and derivatives thereof.

8. A medical device for treating vulnerable plaque comprising;
a medical device selected from the group consisting of catheters, guide wires, vascular stents, micro-particles, electronic leads, probes, sensors, drug depots, transdermal patches, and vascular patches:
said device comprising at least one MMPI composition.

9. The medical device of claim 8 wherein said device is adapted to deliver said MMPI composition to the vulnerable plaque.

10. The medical device according to claim 8 wherein said MMPI is selected from the group consisting of zinc chelators, urea derivatives, caprolactone-based inhibitors, phoshoinamides, piperazines, sulfonamides, tertiary amines, carbamate derivatives, mercaptoalcohols, mecaptoketones, antimicrobial tertracyclines, non-antimicrobial tetracyclines, and derivatives and combinations thereof.

11. The medical device according to claim 10 wherein said antimicrobial tetracycline is selected form the group consisting of tetracycline, doxycycline and minocycline.

12. The medical device according to claim 10 wherein said non-antimicrobial tetracycline CMT-8.

13. The medical device according to claim 10 wherein said zinc chelator is a hydroxamic acid derivative.

14. The medical device according to claim 13 wherein said hydroxamic acid derivative is marimastat or batimastat.

15. The medical device according to claim 8 wherein said MMPI is a naturally occurring tissue inhibitor of metalloproteinases (TIMP) and derivatives thereof.

16. The medical device according to claim 8 further comprising a biocompatible polymer coating.

17. The medical device according to claim 16 wherein said polymer coating is a bioabsorbable polymer selected from the group consisting of poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes and biomolecules such as fibrin, fibrinogen, cellulose, starch, collagen, hyaluronic acid and mixtures thereof.

18. The medical device according to claim 16 wherein said polymer coating is a biostable biocompatible polymer selected from the group consisting of polyurethanes, silicones, and polyesters could be used and other polymers could also be used if they can be dissolved and cured or polymerized on the medical device such as polyolefins, polyisobutylene and ethylene-alphaolefin copolymers, acrylic polymers and copolymers, ethylene-co-vinylacetate, polybutylmethacrylate, vinyl halide polymers and copolymers, such as polyvinyl chloride, polyvinyl ethers, such as polyvinyl methyl ether, polyvinylidene halides, such as polyvinylidene fluoride and polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose and mixtures thereof.

19. A medical device for delivering an MMPI composition to a treatment site within a patient comprising:
at least one MMPI selected from the group consisting of zinc chelators, urea derivatives, caprolactone-based inhibitors, phoshoinamides, piperazines, sulfonamides, tertiary amines, carbamate derivatives, mercaptoalcohols, mecaptoketones, antimicrobial tertracyclines, non-antimicrobial tetracyclines, and derivatives and combinations thereof, and wherein said MMPI is dispersed into a polymer coating applied to said medical device, wherein said polymer coating comprises a base layer of ethylene-co-vinylacetate and polybutylmethacrylate and an outer layer of polybutylmethacrylate.

20. The medical device according to anyone of claims 8, 9, 10, 11, 12, 13, 14, 15, 16, 17, 18 or 19, wherein said medical device is selected from the group consisting of catheters, guide wires, vascular stents, micro-particles, electronic leads, probes, sensors, drug depots, transdermal patches, and vascular patches.

21. A self-expanding vascular stent comprising a coating comprising a MMPI composition, wherein said coated stent is used deliver said MMPI composition to a treatment site within a patient wherein vulnerable plaque has been identified at said treatment site.

22. A self-expanding vascular stent comprising at least one MMPI selected from the group consisting of zinc chelators, urea derivatives, caprolactone-based inhibitors, phoshoinamides, piperazines, sulfonamides, tertiary amines, carbamate derivatives, mercaptoalcohols, mecaptoketones, antimicrobial tertracyclines, non-antimicrobial tetracyclines, and derivatives and combinations thereof, and wherein said MMPI is dispersed into a polymer coating applied to said medical device, wherein said polymer coating comprises poly 2-hydroxyethyl methacrylate (pHEMA) and wherein said pHEMA has a surface comprising ordered methylene chains.

23. A method of delivering a metalloproteinase inhibitor (MMPI) composition to a treatment site within a patent comprising the controlled release of said MMPI composition from a polymer matrix in response to an energy source selected from the group consisting of ultrasound energy, thermal energy and electrical current.
